SEQUENCE LISTING (2 sequences)

SEQ ID NO: 1 - DNA, 995 bp, Artificial Sequence
  Pichia pastoris - GDH2 Promoter
ggacaaccaaagcatccgcctgaagggacaacttgcattcaacggcttcagttggaaacgtcagagctgacctatagtttgctagaaccgttttctctgtttacgtttacgtctcctcaaatttgcgctcggtatgtccttcctaattagcgggaaaagctgttcttagttaatacggagaaagtttcggggttaccgttccgggaagaggaggggtcatctctctcatctcatccaaccattaagtttcttccaaaacttcaggataatcagtttaaccaccgacaggagtcagatttgagattgacagaaagtttttccgtccatttcctcatcttgtcgccgttatcagtcaatctctatggttatctggaatttcttttttcttttaattcatcttctttttatcccgcgcctttggcgttctagctcatctcatgaaaacaaaaccctctcatgttcggataattccagcggctttcactttcagatgacacatagattggactcaaccatggctatctggggtatacggacgttggcaagggcgttaatttttcaggacaaacggaaatgccatggctccagggaaaggcattcctattgcaaacctagaccgtcgaacctctcctatcgcctaccagtcacccagctatccctaggcaactcatctccttcaagcggattgcaacctgctaagccaaattagatctggccacagaaatgccgcaatatttcttggctctcccctccctctggtcttctctcattatcttgcatccttctggcattgtggttttcagaggtttttctaaaaaaataatcacgaccgatggggtctcgatgaggcatctcttaagtcatttttttcccccacaaacagaaaaagcaaaaaactccgtataaatagctccagatcgcccagacgaatctcgcccttgactcaattagtagaaagaaactaaaactaccccccactaaactcccaacccact

SEQ ID NO: 2 - DNA, 1000 bp, Artificial Sequence
  Pichia pastoris - PEPCK promoter
ctacatcggaaccaattttcaattcgccgatacgaaaccggacatcaagttccatcatcatgcaagatgatcatcaggaagagccgtggtcagtcaacactgcaagtgtcaaagagacatcaattattgacattagtaccccggatgaggcttacaatgctcgcgggaaagacgatgcgatacgggttgattcggagggcccttcaaaaagaacaactattctcagccgtctgatggagacacaagattcggacaatgacggaacggagtctgacgtaggggagagttctagtctgataattcttagttgaccagccttgcatcgggttgaacaaacttttgtgggttgggccactttgcgccaacaacacaaggttttcacaccacccggttgtcattaccgcccggaaaactcaatgattgacggagcgtaggttcacggaagtcaagttagagtagtcggagagtttcgggtgataagaatttcccgggtgctttggagtgccaaagtgactgaatcaggaactaaaaacccgggctaacggctgaaggcctgtgttcagtgcactatattgtattctagtctggggtaggttgaagtttggtggattcacgactccggagtgatggagggacaataccgaagatgaggtcttgcgagtggaaagataggttaagaataattaataacattgtatgaaacaagatggggagacaattttagctatcgatccgttatcattggtcgaatgatgacttggaaccttatttgcactttttttgtcaggtgttcgccgaaacttaaagatagttttattgatgtgtgccaaaaatgtggggacaaaaggtcctccacccgccatcccccggtctcatacaaaaaaaaagaaaaatacctctactagtccggggtcaaggcaagtcatgataggcatataaataggggcaggctggccgccattacgaattagattaaccttctcatagattattatccaca

(19) United States
(12) Patent Application Publication
(10) Pub. No.: US 2025/0277248 A1
(43) Pub. Date: Sep. 4, 2025

(54) NUCLEOTIDE SEQUENCE, EXPRESSION VECTORS, RECOMBINANT YEAST CELLS AND APPLICATIONS THEREOF

(71) Applicant: INDIAN INSTITUTE OF SCIENCE, Karnataka (IN)

(72) Inventors: Pundi Narasimhan RANGARAJAN, Bangalore (IN); Trishna DEY, West Bengal (IN)

(73) Assignee: INDIAN INSTITUTE OF SCIENCE, Karnataka (IN)

(21) Appl. No.: 18/266,357

(22) PCT Filed: Dec. 13, 2021
(86) PCT No.: PCT/IB2021/061624

§ 371 Date: Jun. 9, 2023

(30) Foreign Application Priority Data
Dec. 14, 2020 (IN) 202041054332

(51) Int. Cl. C12P 21/00; C12N 1/16; C12N 15/81; C12R 1/84

(52) U.S. Cl. CPC C12P 21/00; C12N 1/165; C12N 15/815; C12R 2001/84

(57) ABSTRACT

The present disclosure relates to nucleotide sequence selected from a group comprising sequence set forth as SEQ ID No. 1 and SEQ ID No. 2. The said nucleotide sequences possess promoter activity. The disclosure further relates to an expression vector comprising the nucleotide sequence, a host cell comprising the said expression vector. The disclosure further relates to method of producing a protein of interest or heterologous protein employing the expression vector. The method provides for simple, economical, and effective means of producing the protein of interest.

### TECHNICAL FIELD

The present disclosure relates to the field of molecular biology. Particularly, the present disclosure relates to promoter selected from a group comprising sequences set forth as SEQ ID No. 1 and SEQ ID No. 2. The disclosure further relates to a vector comprising the promoter(s) and gene of interest. The disclosure further relates to a host cell comprising the vector. The disclosure further relates to a simple, economical and effective method for producing protein of interest employing the promoters and the expression vector.

### BACKGROUND OF THE DISCLOSURE

Yeast expression system can be used to produce proteins, such as enzymes, hormones, receptors, growth factors and vaccine antigens, in part, because Pichia pastoris yeast cells can be grown rapidly to high cell densities in simple and inexpensive media. Being a eukaryote, Pichia pastoris can modify proteins in a manner similar to native proteins in mammals and with a proper signal sequence, the expressed protein can be secreted into the culture medium rendering isolation and purification simple and inexpensive. Drugs, therapeutic proteins and food products produced in yeast expression systems are widely used in the food and pharmaceutical industries. Thus, improvement of yeast expression systems can reduce the cost of isolation and purification of proteins leading to reduction in the cost of human and animal health products as well as food products.

Due to commercial importance of yeast expression systems as well as their importance in academic research to study various biological phenomena, improvement of yeast expression systems is the focus of research and development in industry as well as in academic laboratories. Pichia pastoris has emerged as an important expression system not only for industrial use but also for academic research. Thus, there is a constant need to develop a simple, economical and effective means of producing protein of interest using the yeast system, such as Pichia pastoris.

The present disclosure describes simple, economical and improved means of producing recombinant proteins or heterologous proteins in yeast system.

### SUMMARY OF THE DISCLOSURE

To meet the growing need or demand for the production of recombinant protein or protein of interest in yeast system, such as Pichia pastoris, the Applicant in the present disclosure describes nucleotide sequences selected from a group comprising sequence set forth as SEQ ID No. 1 and SEQ ID No. 2, wherein the said nucleotide sequence possess promoter activity.

In the present disclosure, it is demonstrated that the said nucleotide sequence enhances the production of protein of interest/heterologous protein/recombinant protein in yeast expression system, particularly Pichia pastoris, wherein the said nucleotide sequence is an inducible promoter.

In some embodiments, the present disclosure relates to an expression vector comprising the said nucleotide sequence selected from a group comprising sequence set forth as SEQ ID No. 1 and SEQ ID No. 2 and gene encoding the protein of interest.

In some embodiments, the present disclosure relates to a host cell comprising at least one of the expressing vectors. The said host cell is a recombinant host cell.

In some embodiments, the present disclosure relates to method of producing protein of interest/heterologous protein/recombinant protein employing the said expression vector individually or in combination.

In some embodiments, the present disclosure relates to use of the nucleotide sequence for effective production of protein of interest/recombinant protein.

In some embodiments, the present disclosure relates to use of the expression vector for effective production of protein of interest/recombinant protein.

In some embodiments, the present disclosure relates to method of producing the said expression vector comprising the nucleotide sequence selected from a group comprising sequence set forth as SEQ ID No. 1 and SEQ ID No. 2.

In some embodiments, the present disclosure relates to method of producing the said host cell comprising the expression vector.

In some embodiments, the present disclosure relates to a kit comprising the expression vector comprising the nucleotide sequence selected from a group comprising sequence set forth as SEQ ID No. 1 and SEQ ID No. 2.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with detailed description below, are incorporated in and form part of the specification, and serve to further illustrate the embodiments and explain various principles and advantages, in accordance with the present disclosure where:

FIG. 1 depicts nucleotide sequence of the Pichia pastoris Glutamate dehydrogenase 2 (GDH2) promoter (SEQ ID No. 1) up to 995 bp upstream of the ATG start codon.

FIG. 2 depicts nucleotide sequence of the Pichia pastoris phosphoenolpyruvate carboxykinase (PEPCK) promoter (SEQ ID No. 2) up to 1000 bp upstream of the ATG start codon.

FIG. 3 depicts schematic representation of pIB3 vector obtained from ADDGENE.

FIG. 4 depicts an expression vector constructed by cloning the SEQ ID No. 1-GFP expression cassette (P_{GDH2}-GFP) into the XhoI-HindIII restriction sites of pIB3 vector.

FIG. 5 depicts an expression vector constructed by cloning the SEQ ID No. 2-GFP expression cassette (P_{PEPCK}-GFP) into the KpnI-HindIII restriction sites of pIB3 vector.

FIG. 6 illustrates glutamate-inducible synthesis of GFP employing SEQ ID No. 1 and SEQ ID No. 2, respectively in GS115 Pichia pastoris strain cultured in media containing yeast nitrogen base (YNB) together with glucose (YNBD), glycerol (YNBG), ethanol (YNBE), acetate (YNBA) and glutamate (YNB Glu⁺) by live cell confocal microscopy.

FIG. 7 illustrates carbon source-specific synthesis of His-tagged GDH2 (GDH2^{His}) and Myc-tagged PEPCK (PEPCK^{Myc}) in Pichia pastoris. GDH2 and PEPCK are expressed maximally when the carbon source is glutamate.

FIG. 8 depicts the image of commercially available monosodium glutamate (MSG), a food additive that is used for glutamate-inducible expression of protein of interest or recombinant proteins under the influence of SEQ ID No.1 and SEQ ID No. 2.

FIG. 9 depicts the image of the expression levels of a heterologous protein of interest (GFP) under the influence of SEQ ID No. 1 and SEQ ID No. 2 in cells cultured in presence of glycerol, glutamate and monosodium glutamate (MSG).

FIG. 10 depicts comparison of expression levels of GFP (protein of interest) from methanol-inducible expression vector containing AOXI promoter and glutamate-/MSG-inducible expression vectors containing GDH2 promoter (SEQ ID No. 1) and PEPCK promoter (SEQ ID No. 2). Schematic diagram of expression cassettes (top panel) is shown. Expression of GFP in Pichia pastoris cell lysates was analysed by western blotting using anti-GFP antibodies (middle panel). Quantification of data is shown in the lower panel. Numbers indicate % GFP expression. GFP expression from AOXI promoter is taken as 100%.

FIG. 11 depicts comparison of methanol-inducible GFP expression from AOXI (P_{AOXI}) and MSG-inducible expression from GDH2 (P_{GDH2}) as well as PEPCK (P_{PEPCK}) promoters. GFP was purified from lysates using GST-tagged anti-GFP nanobodies. Cells harboring P_{AOXI}-GFP expression cassette were cultured in YNB medium containing 1% methanol. P_{GDH2}-GFP and P_{PEPCK}-GFP expression cassettes were cultured in YNB medium containing 1% MSG. Cells were lysed and incubated with glutathione beads bound to GST-tagged anti-GFP nanobodies. After washing, proteins bound to glutathione beads were analysed by SDS-PAGE. A BSA standard curve was generated using known amounts of BSA and used for the estimation of amount of GFP expressed from P_{AOXI}, P_{GDH2} and P_{PEPCK}. GFP expression from AOXI promoter is taken as 100%.

FIG. 12 Illustrates schematic description of glutamate/MSG-inducible pTR1-IX, pTR1-SX, pTR2-IX and pTR2-SX vectors. IX vectors and SX vectors are meant for intracellular and extracellular expression of recombinant proteins, respectively. pTR1 and pTR2 vectors contain GDH2 (SEQ ID No. 1) and PEPCK (SEQ ID No. 2) promoters respectively. Presence of selection markers, such as HIS4, HYG^{R} and ZEO^{R} are also indicated.

FIG. 13 depicts schematic representation of expression vectors used for glutamate-/MSG-inducible expression of heterologous proteins from Pichia pastoris having SEQ ID No. 1 (GDH2 promoter) and SEQ ID No. 2 (PEPCK promoter), respectively. pTR1-IX and pTR2-IX expresses protein of interest intracellularly employing SEQ ID No. 1 and SEQ ID NO. 2, respectively. pTR1-SX and pTR2-SX are used for secretion of the protein of interest into culture medium employing SEQ ID No. 1 and SEQ ID NO. 2, respectively. MCS in the vector stands for multiple cloning site, HIS4 gene encodes histidinol dehydrogenase involved in histidine biosynthesis and serves as an auxotrophic selection marker. HYG^{R} and ZEO^{R} confer hygromycin and zeocin resistance in both yeast and bacteria. IX stands for intracellular expression of recombinant proteins; and SX stand for extracellular expression of recombinant proteins.

FIG. 14A and C depicts MSG-inducible expression of His-tagged human actin B (HsActinB-His) and His-tagged SARS COV-2-receptor binding domain (RBD-His) from PEPCK promoters of pTR2-IX^{HIS4,HYGR} (FIG. 14A) and pTR2-SX^{HIS4,HYGR} (FIG. 14C) vectors respectively. B and D illustrate gel image depicting expression of HsActinB-His and RBD-His.

FIG. 15A, B and C depicts glutamate inducible vectors for expression of GFP, wherein the vector under A comprises SEQ ID NO. 1, vector and B and C comprises SEQ ID NO. 2; D, E and F illustrates improved expression of GFP in P. pastoris when the cells were co-transformed with vectors pTR1-IX^{HIS4,HYGR}-GFP (a) and pTR2-IX^{ZEOR}-GFP (b) or pTR2-IX^{ZEOR}-GFP (b) and pTR2-IX ^{HIS4,HYGR}-GFP (c). MSG-inducible expression of GFP from recombinant P. pastoris strain harbouring plasmids b+c is higher than methanol-inducible expression of GFP in the P. pastoris strain harbouring P^{AOXI}-GFP.

FIG. 16A and B depicts expression vector pTR2-SX^{HIS4,HYGR} and pTR2-SX^{ZEOR} for expression of His-tagged SARSCOV-2-RBD (RBD-His) under the influence of SEQ ID NO. 2; and C and D illustrates expression of His-tagged SARSCOV-2-RBD (RBD-His) in a cell co-transformed with vectors depicted in A and B.

### DETAILED DESCRIPTION OF THE DISCLOSURE

Unless otherwise defined, all terms used in the disclosure, including technical and scientific terms, have meaning as commonly understood by one of ordinary skill in the art to which this invention belongs. By means of further guidance, term definitions are included to better understand the present disclosure.

As used herein, the singular forms ‘a’, ‘an’, and ‘the’ include both singular and plural referents unless the contest clearly dictates otherwise.

The term ‘comprising’, ‘comprises’ or ‘comprised of’ as used herein are synonymous with ‘including’, ‘includes’, ‘containing’ or ‘contains’ and are inclusive or open-ended and do not exclude additional, non-recited members, elements or method steps.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of ±10% or less, preferably ±5% or less, more preferably ±1% or less and still more preferably ±0.1% or less of and from the specified value, insofar such variations are appropriate to perform the present disclosure. It is to be understood that the value to which the modifier ‘about’ refers is itself also specifically, and preferably disclosed.

The term ‘polynucleotide’ or ‘nucleotide sequence’ or ‘nucleic acid molecule’ is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as DNA/RNA hybrid.

The terms ‘recombinant nucleic acid’ or ‘recombinant nucleic acid molecule’ as used herein generally refer to nucleic acid molecules (such as, e.g., DNA, cDNA or RNA molecules) comprising segments generated and/or joined together using recombinant DNA technology, such as for example molecular cloning and nucleic acid amplification. Usually, a recombinant nucleic acid molecule may comprise one or more non-naturally occurring sequences, and/or may comprise segments corresponding to naturally occurring sequences that are not positioned as they would be positioned in a source genome which has not been modified. When a recombinant nucleic acid molecule replicates in the host organism (host cell) into which it has been introduced, the progeny nucleic acid molecule(s) are also encompassed within the term “recombinant nucleic acid molecule”. The recombinant nucleic acid molecule can be stably integrated into the genome of a host organism, such as for example integrated at one or more random positions or integrated in a targeted manner, such as, e.g., by means of homologous recombination, or the recombinant nucleic acid molecule can be present as or comprised within an extra-chromosomal element (plasmid), wherein the latter may be auto-replicating.

The term ‘recombinant protein’ or ‘recombinant polypeptide’ or ‘protein of interest’ or ‘polypeptide of interest’ as used herein refers to a polypeptide or protein produced by a host organism (host cell) through the expression of a recombinant nucleic acid molecule, which has been introduced into said host organism and which comprises a sequence encoding said polypeptide or protein.

The term ‘promoter’ used herein includes transcriptional regulatory sequences required for accurate transcription initiation and where applicable accurate spatial and/or temporal control of gene expression or its response to, e.g., internal or external (e.g., exogenous) stimuli. More particularly, ‘promoter’ may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. The promoters contemplated herein are inducible. The mRNA synthesized from the promoters described herein may contain 5′ untranslated regions (5′ UTRs) which promote efficient translation, leading high levels of synthesis of proteins of interest in cells cultured in specific growth media.

The term ‘construct’ or ‘expression construct’ or ‘expression vector’ used herein mean any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, capable of genomic integration or autonomous replication, comprising a nucleic acid molecule where one or more nucleic acid sequences has been linked in a functionally operative manner, i.e. operably linked. As used herein ‘vector’ means any plasmid, or other vector, in double-stranded or single-stranded form or in linear or circular form that can transform a host cell by integration into the host cell genome or by existing extrachromosomal sequence (e.g., an autonomously replicating plasmid). In other words, a vector (e.g., expression vector or expression cassette) is a nucleic acid construct used to transform a host cell for expression of a protein, polypeptide, or peptide and the vector is not found in nature in the host cell it transforms.

The present disclosure relates to nucleic acid sequence selected from a group comprising sequence set forth as SEQ ID No. 1 and SEQ ID No. 2.

In some embodiments of the present disclosure, SEQ ID No. 1 is Glutamate dehydrogenase 2 (GDH2).

In some embodiments of the present disclosure, SEQ ID No. 2 is phosphoenolpyruvate carboxykinase (PEPCK).

In some embodiments, the present disclosure relates to nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 1

In some embodiments, the present disclosure relates to nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 2.

In some embodiments of the present disclosure, the nucleic acid sequence set forth as SEQ ID No.1 and SEQ ID No.2, independently possess promoter activity capable of influencing effective production of protein of interest or recombinant protein or heterologous protein.

In some embodiments of the present disclosure, the nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 1, possess promoter activity capable of influencing effective production of protein of interest or recombinant protein.

In some embodiments of the present disclosure, the nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 2, possess promoter activity capable of influencing effective production of protein of interest or recombinant protein.

In some embodiments of the present disclosure, the nucleic acid sequence selected from a group comprising sequence set forth as SEQ ID No. 1 and SEQ ID No. 2 are obtained from Komagataella phaffii GS115 strain. This strain has mutation in the HIS4 gene, as a result, it is defective in histidine biosynthesis. P. pastoris expression strains employed in the present disclosure are derivatives of NRRL-Y 11430 (Northern Regional Research Laboratories, Peoria, Illinois, USA).

In some embodiments of the present disclosure, the nucleic acid sequence selected from a group comprising sequence set forth as SEQ ID No. 1 and SEQ ID No. 2 is inducible promoter.

In some embodiments of the present disclosure, the nucleic acid sequence selected from a group comprising sequence set forth as SEQ ID No. 1 and SEQ ID No. 2 is glutamate inducible promoter.

In some embodiments of the present disclosure, the nucleic acid sequence selected from a group comprising sequence set forth as SEQ ID No. 1 and SEQ ID No. 2 is monosodium glutamate inducible promoter.

In some embodiments of the present disclosure, the nucleic acid sequence selected from a group comprising sequence set forth as SEQ ID No. 1 and SEQ ID No. 2 having promoter activity enhances the production of protein of interest or recombinant protein in the presence of substrate including but not limited to glutamate and monosodium glutamate.

In some embodiments of the present disclosure, the nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 1, having promoter activity enhances the production of protein of interest or recombinant protein in the presence of carbon source including but not limited to glutamate and monosodium glutamate.

In some embodiments of the present disclosure, the nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 2, having promoter activity enhances the production of protein of interest or recombinant protein in the presence of carbon source including but not limited to glutamate and monosodium glutamate.

The inventors have particularly identified that the nucleic acid sequence selected from a group comprising sequence set forth as SEQ ID No. 1 and SEQ ID No. 2 enables formation of mRNA of gene of interest under carbon sources, such as glucose, glycerol, ethanol and acetate. However, the produced mRNA will not be translated into protein unless the said carbon source is replaced by other carbon source, such as glutamate, monosodium glutamate and combination thereof. Thus, the expression of protein of interest can be particularly controlled under the influence of sequence set forth as SEQ ID No. 1 or SEQ ID No 2 or combination thereof.

In some embodiments of the present disclosure, the nucleic acid sequence selected from a group comprising sequence set forth as SEQ ID No. 1 and SEQ ID No. 2 having promoter activity enhances the production of protein of interest or recombinant protein in microorganisms which can utilize amino acids such as glutamate, aspartate as the sole source of carbon.

In some embodiments of the present disclosure, the nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 1, having promoter activity enhances the production of protein of interest or recombinant protein in microorganisms which can utilize amino acids such as glutamate, aspartate as the sole source of carbon.

In some embodiments of the present disclosure, the nucleic acid sequence having at least 90%, at least 91%, at least 92%, at least 93%, at least 94%, at least 95%, at least 96%, at least 97%, at least 98% or at least 99% sequence identity to nucleic acid sequence set forth as SEQ ID No. 2, having promoter activity enhances the production of protein of interest or recombinant protein in microorganisms which can utilize glutamate, aspartate as the sole source of carbon.

In some embodiments, the present disclosure relates to expression vector comprising the said nucleic acid sequence selected from a group comprising sequence set forth as SEQ ID No. 1 and SEQ ID No. 2 and the gene of interest.

In some embodiments of the present disclosure, the expression vector comprising the nucleic acid sequence selected from a group comprising sequence set forth as SEQ ID No. 1 and SEQ ID No.2, operably linked to gene of interest, wherein the gene of interest is a heterologous gene encoding the protein of interest.

In some embodiments of the present disclosure, the gene encoding protein of interest is cloned downstream of the SEQ ID No. 1 and/or SEQ ID No. 2 in the expression vector. In some embodiments of the present disclosure, the expression vector comprising the nucleic acid sequence set forth as SEQ ID No. 1 includes but is not limited to pTR1-IX and pTR1-SX.

In some embodiments of the present disclosure, the expression vector comprising the nucleic acid sequence set forth as SEQ ID No. 2 includes but is not limited to pTR2-IX and pTR2-SX.

In some embodiments of the present disclosure, cloning of the gene of interest into the expression vector including but is not limited to pTR1-IX and pTR2-IX, results in intracellular expression of the encoded protein.

In some embodiments of the present disclosure, cloning of the gene of interest into the expression vectors including but is not limited to pTR1-SX and pTR2-SX, results in secretion of the encoded protein into the culture medium.

In an embodiment of the present disclosure, FIG. 4 illustrates the expression vector comprising SEQ ID No. 1. The expression vector illustrates (as an example) cloning of GFP (protein of interest) under the influence of the SEQ ID No. 1.

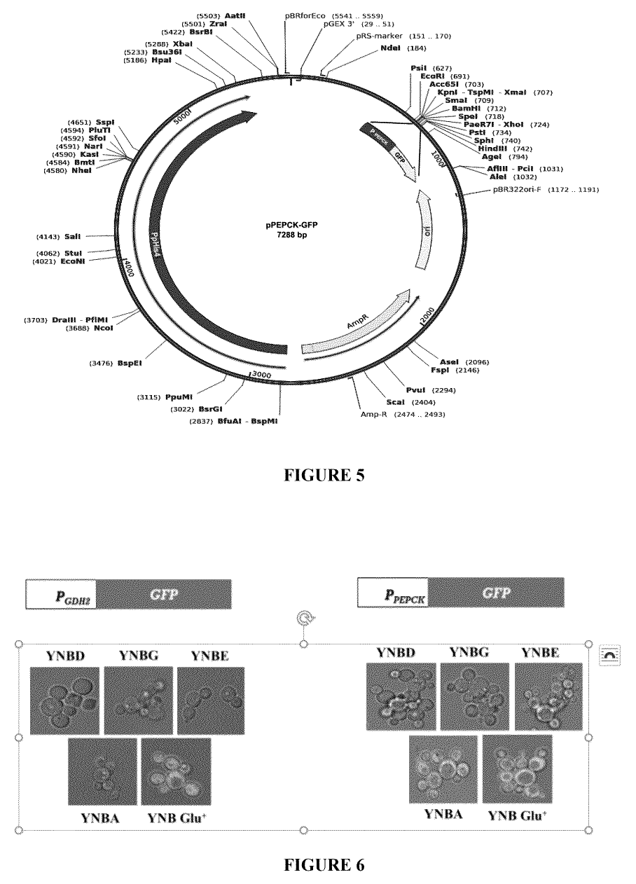

In an embodiment of the present disclosure, FIG. 5 illustrates the expression vector comprising SEQ ID No. 2. The expression vector illustrates (as an example) cloning of GFP (protein of interest) under the influence of the SEQ ID No. 2.

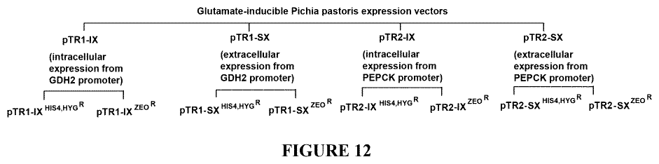

In an embodiment of the present disclosure, the FIG. 12 provides a schematic representation of the vectors pTR1-IX, pTR1-SX, pTR2-IX and pTR2-SX meant for intracellular and extracellular expression of recombination proteins. pTR1 and pTR2vectors comprises GDH2 (SEQ ID No. 1) and PEPCK (SEQ ID No. 2) promoters respectively. HIS4, HYG^{R} and ZEO^{R} in the vector are selection markers.

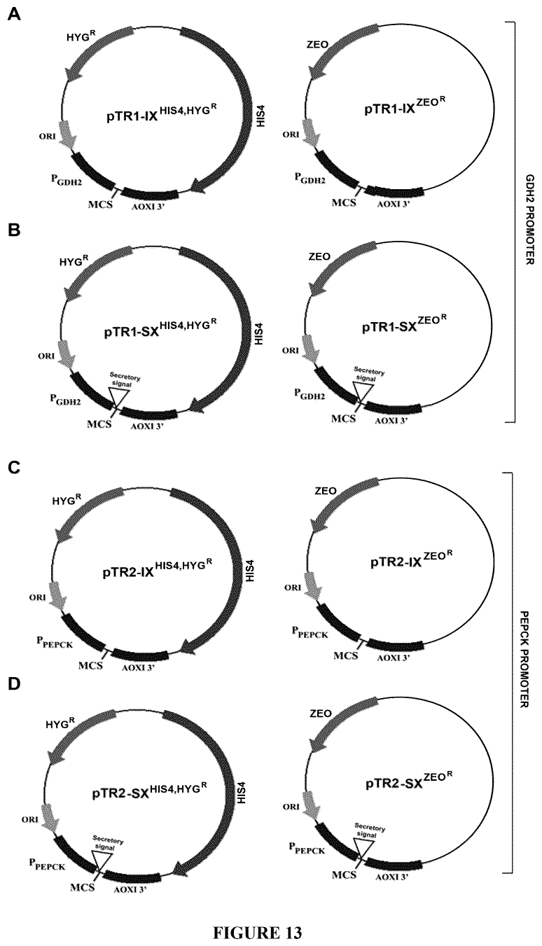

In an embodiment of the present disclosure, the FIG. 13 illustrates expression vectors pTR1-IX, pTR2-IX, pTR1-SX and pTR2-SX comprising nucleotide sequence set forth as SEQ ID NO. 1 and SEQ ID NO. 2 and suitable selection markers, such as HIS4, HYG^{R}and ZEO^{R}.

In some embodiments of the present disclosure, the expression vector is a yeast expression vector capable of producing effective amounts of heterologous protein or protein of interest intracellularly or secreted into the yeast cells so that the protein can be conveniently isolated and purified.

In some embodiments of the present disclosure, the expression vector comprising the nucleotide sequence selected from a group comprising sequence set forth as SEQ ID No. 1 and SEQ ID No. 2 is also compatible with other expression system apart from yeast.

In some embodiments of the present disclosure, the expression vector comprises selection marker, a cloning site, such as multiple cloning site, an enhancer sequence, a termination sequence and a signal peptide sequence.

In some embodiments of the present disclosure, the expression vector comprises restriction endonuclease cleavage sites for the insertion of DNA fragments (e.g., one or more cloning sites and/or a multiple cloning site), and genetic markers for selection of transformants can include a selection marker that allows a transformed host cell to grow on a medium devoid of a necessary nutrient that cannot be produced by a deficient strain (For examples, Pichia pastoris HIS4 encoding Histidinol dehydrogenase; Pichia pastoris MET2 encoding homoserine-O-transacetylase), a selection marker that encodes an enzyme for which chromogenic substrates are known, or a selection marker that provides resistance to a drug includes but is not limited to G418, Nourseothricin (Nat), Zeocin, Blasticidin or Hygromycin.

In some embodiments of the present disclosure, the expression vector has a terminator sequence for transcription termination (for e.g., AOXI gene).

In some embodiments of the present disclosure, the expression vector has a 3′ untranslated region (3′ UTR) downstream from the protein (protein of interest) coding sequence with a polyadenylation site. As used herein, 3′ UTR means nucleotide sequences that are not translated into protein and are located downstream from a coding sequence for a protein. Typically, a 3′ UTR includes regulatory sequence for mRNA processing.

In some embodiments of the present disclosure, the expression vector has a 5′ untranslated region (5′ UTR) upstream of the protein coding sequence which regulates translation such that proteins are synthesized from pre-existing mRNAs only when cells are cultured in growth media having specific carbon source. The inventors have particularly identified that the expression vector comprising the nucleic acid sequence selected from a group comprising sequence set forth as SEQ ID No. 1 and SEQ ID No. 2 enables formation of mRNA of gene of interest under carbon sources, such as glucose and glycerol. However, the produced mRNA will not be translated into protein unless the carbon source such as glucose, ethanol, acetate and glycerol are replaced by carbon source such as glutamate, monosodium glutamate and combination thereof. Thus, the expression of protein of interest can be particularly controlled by the expression vector comprising the sequence set forth as SEQ ID No. 1 or SEQ ID No 2 or combination thereof.

In some embodiments of the present disclosure, the expression vector has an origin of replication for use in synthesizing and amplifying the vector in organism including but not limited to Pichia pastoris, Yeasts such as, Scheffersomyces stipitis (also known as Pichia stipitis), Candida albicans, Candida maltosa, Candida shehatae, Candida glabrata, Candida reukaufii, Candida utilis, Debaryomyces hansenii, Kluyveromyces lactis, Kluyveromyces marxianus, Lodderomyces elongisporus, Meyerozyma guilliermondii, Pichia capsulata, Yarrowia lipolytica, Rhodotorula rubra, and Trichosporon beigelii.

In some embodiments of the present disclosure, the expression vector is a vector that replicates autonomously or integrates into the host cell genome.

In some embodiments of the present disclosure, the expression vector is circularized or linearized (i.e., digested with a restriction enzyme so that a gene of interest can easily be cloned into the expression vector).

In some embodiments, the present disclosure relates to a method of producing the said expression vector.

In some embodiments of the present disclosure, the method of producing the expression vector comprises—
combining the nucleotide sequence selected from a group comprising sequence set forth as SEQ ID No. 1 and SEQ ID No. 2 and a protein of interest to obtain expression cassette; and
cloning the expression cassette into a vector to obtain the expression vector.

In some embodiments of the present disclosure, the method of producing the expression vector comprises obtaining the nucleotide sequences selected from a group comprising sequence set forth as SEQ ID No. 1 and SEQ ID No. 2 by polymerase chain reaction and joining them, respectively to the gene of interest by polymerase chain reaction to obtain the expression cassette. The expression cassette is cloned into the yeast vector including but not limited to pIB3 using restriction enzymes including but not limited to XhoI, KpnI and NotI to obtain the expression vector.

In some embodiments, the present disclosure relates to host cell comprising the above-described expression vector. The said host cell is a recombinant host cell.

In some embodiments of the present disclosure, the host cell is prokaryotic organism or eukaryotic organism.

In some embodiments of the present disclosure, the host cell includes but is not limited to Pichia pastoris, Scheffersomyces stipitis (also known as Pichia stipitis), Candida albicans, Candida maltosa, Candida shehatae, Candida glabrata, Candida reukaufii, Candida utilis, Debaryomyces hansenii, Kluyveromyces lactis, Kluyveromyces marxianus, Lodderomyces elongisporus, Meyerozyma guilliermondii, Pichia capsulata, Yarrowia lipolytica, Rhodotorula rubra, and Trichosporon beigelii which can utilize glutamate as carbon source may also serve as host cells.

In some embodiments of the present disclosure, the host cell is capable of effectively producing protein of interest or recombinant protein or heterologous protein under the influence of the nucleotide sequence selected from a group comprising sequence set forth as SEQ ID No. 1 and SEQ ID No. 2 present in the expression vector.

In some embodiments of the present disclosure, the host cell comprises at least one expression vector described above.

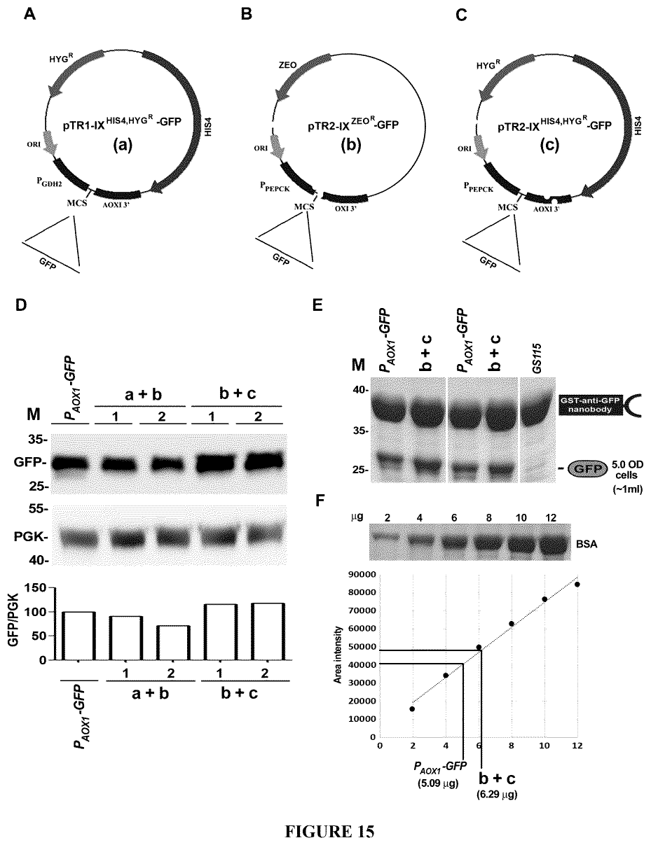

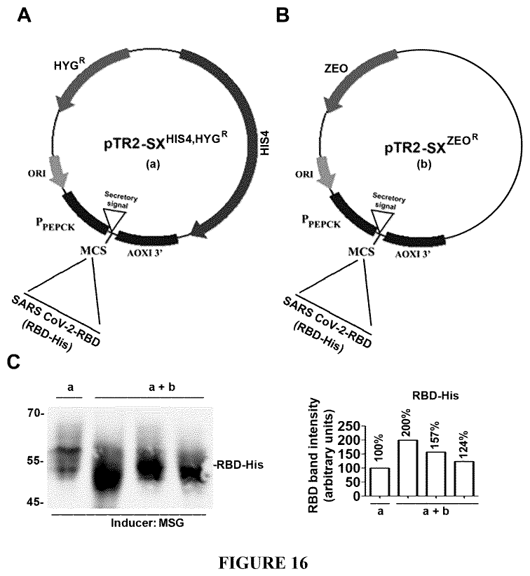

In some embodiments of the present disclosure, the host cell comprises combination of expression vector comprising SEQ ID No. 1 and/or SEQ ID No. 2 having same or distinct selection marker. FIGS. 15 and 16 describes expression of protein of interest employing combination of expression vectors.

In some embodiments of the present disclosure, the host cell comprises the expression vector comprising SEQ ID No. 1 operably linked to gene of interest.

In some embodiments of the present disclosure, the host cell comprises the expression vector comprising SEQ ID No. 2 operably linked to gene of interest.

In some embodiments of the present disclosure, the host cell comprises the expression vector comprising SEQ ID No. 1 operably linked to gene of interest and the expression vector comprising SEQ ID No. 2 operably linked to gene of interest.

In an embodiment of the present disclosure, the FIG. 15 describes expression of protein of interest (for e.g., GFP) in a host cell comprising combination of the expression vector described above. The FIG. 15 describes expression vectors a, b and c. The data in the figure demonstrates that combination of expression vectors a+b and/or b+c in the host cell provides for improved expression of the protein of interest. The data in the FIG. 15 particularly demonstrates that MSG-inducible expression of GFP (heterologous protein) from recombinant P. pastoris strain harbouring plasmids b+c is higher than methanol-inducible expression of GFP in the P. pastoris strain harbouring PAOXI-GFP.

In an embodiment of the present disclosure, the FIG. 16 describes expression of protein of interest (for e.g., SARS COV2-RBD) in a host cell comprising combination of the expression vector described above. The FIG. 16 describes expression vectors (a) and (b). The data in the figure demonstrates that combination of expression vectors a+b in the host cell provides for improved expression of the protein of interest.

In some embodiments, the present disclosure relates to method of producing the recombinant host cell comprising the said expression vector.

In some embodiments of the present disclosure, the method of producing the host cell (recombinant host cell) comprising the expression vector comprises-transforming a host cell with the expression vector to obtain recombinant host cell.

In some embodiments of the present disclosure, the method of producing the recombinant host cell comprises—
preparing an expression vector comprising the nucleotide sequence selected from a group comprising sequence set forth as SEQ ID No. 1 and SEQ ID No. 2 operably linked to a protein of interest; and
transforming a cell with the said expression vector to obtain recombinant host cell.

In some embodiments of the present disclosure, the method of producing the recombinant host cell comprises-creating a stressed environment for the host cell to enable uptake of the expression vector.

In some embodiments of the present disclosure, the method of producing the recombinant host cell comprises selecting the host cell effectively transformed with the expression vector.

In some embodiments, the present disclosure relates to method of producing heterologous protein or protein of interest.

In some embodiments of the present disclosure, the method of producing the heterologous protein or protein of interest is simple, economical and effective.

In some embodiments of the present disclosure, the method of producing the heterologous protein or protein of interest comprises-allowing growth of the recombinant host cell described above in a suitable culture medium.

In some embodiments of the present disclosure, the method of producing the heterologous protein or protein of interest comprises:
allowing growth of the recombinant host cell described above in a culture medium having carbon source selected from a group comprising glucose glycerol, ethanol and acetate; and
replacing the carbon source with component (other carbon source) selected from a group comprising glutamate, monosodium glutamate and combination thereof for producing the heterologous protein.

In some embodiments of the present disclosure, the method of producing heterologous protein or protein of interest comprises—
preparing the expression vector comprising the nucleotide sequence selected from a group comprising sequence set forth as SEQ ID No. 1 and SEQ ID No. 2 operably linked to a gene of interest;
transforming a cell with said expression vector;
selecting transformed cell;
culturing said selected transformed cell in a suitable culture medium under conditions permitting growth but not production of the protein of interest or the heterologous protein;
further, culturing said selected transformed cell in a suitable culture medium under conditions permitting production of the protein of interest or the heterologous protein but not efficient growth of the cell;
recovering the produced protein; and
optionally purifying the said recovered protein.

In some embodiments of the present disclosure, the transformed cell comprises the expression vector comprising SEQ ID No. 1 operably linked to gene of interest.

In some embodiments of the present disclosure, the transformed cell comprises the expression vector comprising SEQ ID No. 2 operably linked to gene of interest.

In some embodiments of the present disclosure, the transformed cell comprises the expression vector comprising SEQ ID No. 1 operably linked to gene of interest and the expression vector comprising SEQ ID No. 2 operably linked to gene of interest, wherein the gene of interest in both the expression vector can be same or different.

In some embodiments of the present disclosure, culturing comprises growing the transformed cells in media containing compounds including but not limited to glucose, glycerol, methanol, acetate, ethanol, fatty acids, amino acids such as glutamate, aspartate and proline.

In some embodiments of the present disclosure, the media that permits only growth of the cell and not permit production of the protein of interest during culturing includes but it is not limited to yeast nitrogen base (YNB), biotin, sodium chloride, ammonium sulphate, calcium sulphate, potassium sulphate, magnesium sulphate, phosphoric acid, glycerol glucose, trace elements, ammonium hydroxide. This media that permits only growth of the transformed cell additionally comprises carbon source selected from a group comprising glucose, glycerol, ethanol, acetate, and combination thereof.

In some embodiments of the present disclosure, the media that permits production of protein of interest and not growth (not efficient growth) of the cell during culturing includes but it is not limited to yeast nitrogen base, biotin, sodium chloride, ammonium sulphate, calcium sulphate, potassium sulphate, magnesium sulphate, phosphoric acid, amino acids, methanol, trace elements, ammonium hydroxide. This media that permits production of protein of interest additionally comprises carbon source selected from a group comprising glutamate, monosodium glutamate and combination thereof. This carbon source is absent in the media mentioned above that only permits growth of the transformed cell.

In some embodiments of the present disclosure, culturing the transformed cell is carried out under non-inducible or non-permissive conditions, using carbon source other than glutamate or monosodium glutamate including but not limited to glucose and glycerol. Thereafter, the carbon source is changed to glutamic acid or monosodium glutamate which permits for maximal expression of gene of interest for producing the protein of interest.

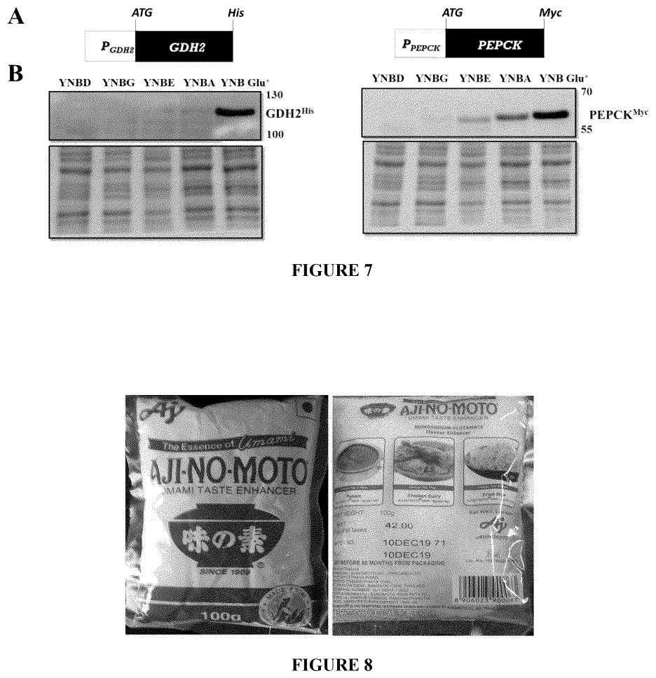

The method of present disclosure for producing the protein of interest or the heterologous protein production at a controlled and at an improved rate due to the controller imposed by the method in producing the protein which is attributed to the presence of nucleotide sequence selected from a group comprising SEQ ID No. 1 and SEQ ID No. 2 in the expression vector of the recombinant host cell (transformed cell) alongside the protein of interest or the heterologous protein. The gel image in the FIG. 7 of the present disclosure particularly demonstrates improved expression of the protein of interest, for e.g., GDH2 and PEPCK from the recombinant host in presence of glutamate as carbon source in the culture medium when compared to other carbon sources, such as glucose glycerol, ethanol and acetate in the culture medium.

In some embodiments of the present disclosure, recovering the protein of interest/heterologous protein comprises harvesting the cultured cells by technique including but not limited to centrifugation, followed by processing the extract/pellet obtained after harvesting.

In some embodiments of the present disclosure, the purification of the protein involves subjecting the protein to techniques including but not limited to ammonium sulfate or ethanol precipitation, acid extraction, gel filtration, anion or cation exchange chromatography, DEAE-sepharose column chromatography, hydroxylapatite chromatography, lectin chromatography, affinity chromatography, solvent-solvent extraction, ultrafiltration and HPLC.

In some embodiments of the present disclosure, when the protein is not secreted into the culture medium, the recombinant host cell can be lysed for example by sonication, by vortexing in presence of glass beads, heat or chemical treatment and the homogenate is centrifuged to remove cell debris. The supernatant can then be subjected to techniques including but not limited to ammonium sulfate precipitation, gel filtration, ion exchange chromatography, DEAE-sepharose column chromatography, affinity chromatography, solvent-solvent extraction, ultrafiltration and High Performance Liquid Chromatography (HPLC).

In some embodiments of the present disclosure, the gene of interest encodes proteins selected from a group comprising toxin, antibody, hormone, enzyme, growth factor, cytokine, structural protein, immunogenic protein, vaccine protein, cytokine, fusion protein, chimeric protein, membrane protein, non-membrane protein and cell signalling protein.

In some embodiments of the present disclosure, the toxins can be proteins such as, for example, botulinum toxin or verotoxin-1, and after preparation using the methods, isolated nucleic acids, expression vectors, host cells, and DNA constructs described herein, the toxins can be modified using a targeting agent so that they are directed specifically to diseased cells.

In some embodiments of the present disclosure, the antibody can be a humanized antibody, an antibody that is not humanized, a nanobody, or an antibody fragment, such as a Fab fragment of an antibody or a single-chain antibody.

In some embodiments of the present disclosure, the hormone can be, for example, a gonadotropin, an adrenocorticotrophic hormone, a growth hormone, vasopressin, oxytocin, somatostatin, gastrin, or leptin.

In some embodiments of the present disclosure, the growth factor can be insulin, epidermal growth factor, fibroblast growth factor, vascular endothelial growth factor, erythropoietin, platelet-derived growth factor, thrombopoietin, or a bone morphogenic protein. In one aspect, the cytokine can be IL-2, IFN-α, IFN-γ, or GM-CSF.

In some embodiments of the present disclosure, the vaccine protein can be any suitable vaccine proteins that are immunogenic in a patient or an animal, including, but not limited to, HPV proteins (e.g., HPV 16 and HPV 18), Hepatitis B virus surface antigen, proteins derived from corona viruses and tetanus vaccine proteins, as examples.

In some embodiments of the present disclosure, the enzymes can be, for example, enzymes for animal feeds as discussed herein, acetylcholinesterase, or cyclooxygenase, or any other useful enzyme that can be expressed in organism including but not limited to yeast.

In some embodiments of the present disclosure, cellular proteins such as actin, netrins, actin-binding proteins, or myosin can be expressed. Cell signaling proteins such as ras proteins, kinases, the ErbB2 protein (the Her-2 receptor) can be expressed using the methods, isolated nucleic acids, expression vectors, host cells, and DNA constructs described herein.

In some embodiments, the present disclosure relates to a kit.

In some embodiments of the present disclosure, the kit comprises the expression vector comprising the nucleotide sequence selected from a group comprising sequence set forth as SEQ ID No. 1 and SEQ ID No. 2.

In some embodiments of the present disclosure, the kit comprises the expression vector comprising the nucleotide sequence selected from a group comprising sequence set forth as SEQ ID No. 1 and SEQ ID No. 2, restriction endonuclease, ligase, buffer, instruction for use and any other components suitable for use in a kit for making and using the expression vector.

In some embodiments of the present disclosure, the kit comprises the expression vector comprising the nucleotide sequence set forth as SEQ ID No. 1, restriction endonuclease, ligase, buffer, instruction for use and any other components suitable for use in a kit for making and using the expression vector.

In some embodiments of the present disclosure, the kit comprises the expression vector comprising the nucleotide sequence set forth as SEQ ID No. 2, restriction endonuclease, ligase, buffer, instruction for use and any other components suitable for use in a kit for making and using the expression vector.

In some embodiments of the present disclosure, the kit comprises an expression vector and a control ORF encoding a marker or control gene for expression (e.g., an ORF encoding a LacZ-fragment or GFP) for use as a control to show that the expression vector is competent to be ligated and to be used with a gene of interest.

In some embodiments of the present disclosure, the kit includes circular expression vector.

In some embodiments of the present disclosure, the kit includes linear expression vector that is digested with a restriction enzyme so that it is ready to clone a gene of interest.

In some embodiments of the present disclosure, the kit includes circular or linear expression vector and a control ORF encoding a marker or control gene for expression (for e.g., an ORF encoding GFP or LacZ fragment) for use as a control in showing that the expression vector can be ligated with a gene of interest, and/or to show that the host cell is competent for transformation.

In some embodiments of the present disclosure, the kit can contain multiple different expression vectors described above. The multiple different expression vectors can contain the same promoter (SEQ ID No. 1 or SEQ ID No. 2), but different selectable markers, such as genes for enzymes involved in biosynthesis of amino acids, purines, pyrimidines (for e.g., Pichia pastoris HIS4 encoding Histidinol dehydrogenase; Pichia pastoris MET2 encoding homoserine-O-transacetylase.), genes conferring resistance to the drugs such as G418, Nourseothricin (Nat), Zeocin, Blasticidin, or Hygromycin.

In some embodiments of the present disclosure, the kit alongside the expression vector described above can include components useful for transformation of yeast cells, restriction enzymes for incorporating a gene encoding protein of interest into the expression vector, ligases, components for purification of the expression vector constructs, buffers (e.g., ligation buffer), instruction for use (for e.g., to facilitate cloning) and any other components suitable for use in the kit for making and using the expression vector described herein.

In some embodiments of the present disclosure, the kit enables effective production of protein of interest or heterologous protein in simple and economical manner.

In some embodiments, the present disclosure relates to use of the nucleotide sequence selected from a group comprising sequence set forth as SEQ ID No. 1 and SEQ ID No. 2.

In some embodiments of the present disclosure relates to use of the nucleotide sequence selected from a group comprising sequence set forth as SEQ ID No. 1 and SEQ ID No. 2 for cloning of the gene encoding protein of interest.

In some embodiments of the present disclosure, the use of the nucleotide sequence selected from a group comprising sequence set forth as SEQ ID No. 1 and SEQ ID No.2 enables simple, economical and effective production of protein of interest or heterologous protein.

In some embodiments of the present disclosure, the use of the nucleotide sequence selected from a group comprising sequence set forth as SEQ ID No. 1 and SEQ ID No. 2 enables production of the protein of interest from a gene encoding the protein of interest in a culture medium having carbon source selected from a group comprising glutamate, monosodium glutamate and combination thereof.

In some embodiments, the present disclosure relates to use of the expression vector described above.

In some embodiments of the present disclosure, the use of the expression vector enables simple, economical and effective production of protein of interest or heterologous protein.

In some embodiments of the present disclosure, the use of the nucleotide sequence and the use of the expression vector provides for improved production of protein of interest or heterologous protein in organism including but not limited to yeast.

In some embodiments, the present disclosure relates to use of the expression vector described above for transforming a host cell for expressing heterologous protein at an improved rate.

The present disclosure provides for following advantages-i)
i) Introduction of single or multiple copies of the expression vector described above (for e.g., pTR1 and pTR2) into the same Pichia pastoris strain can result in high levels of glutamate-inducible expression of protein of interest or heterologous protein than that from either of them alone.
ii) Monosodium glutamate (MSG) is non-toxic and certified as Generally Regarded as Safe (GRAS) by USFDA and is used as common food additive globally. Thus, the described method of producing protein of interest or heterologous protein using MSG is simple, economical and efficient.
iii) Metabolism of glutamate or MSG does not produce toxic molecules such as hydrogen peroxide as observed during methanol-inducible expression of recombinant and therefore oxidative stress and consequent degradation of recombinant proteins is minimal.
iv) The availability of two glutamate-inducible promoters (SEQ ID No. 1 and SEQ ID No. 2) of variable efficacy provides a unique opportunity for differential expression of two proteins in the same cell. This is of great utility during metabolic engineering of yeast cells when there is a need to express two different enzymes of a metabolic pathway at variable levels.
v) Co-transformation of Pichia pastoris with different combinations of the expression vector (for e.g., pTRI and pTR2) carrying different selection markers but the same gene of interest can further enhance glutamate-inducible expression of the gene of interest.

The description further relates to numbered embodiments describing the feature of the present disclosure—
1. A nucleotide sequence selected from a group comprising-- sequence having at least 90% identity to sequence set forth as SEQ ID No. 1; and sequence having at least 90% identity to sequence set forth as SEQ ID No. 2.
2. The nucleotide sequence of embodiment 1, wherein the SEQ ID No. 1 is glutamate dehydrogenase 2 (GDH2) gene, having promoter activity.
3. The nucleotide sequence of embodiment 2, wherein the SEQ ID No. 2 is phosphoenolpyruvate carboxykinase (PEPCK) gene, having promoter activity.
4. The nucleotide sequence of embodiment 1, wherein the sequence is at least 91%, at least 92%, at least 93%, at least 94%, at least 95%, at least 96%, at least 97%, at least 98% or at least 99% identical to the sequence set forth as SEQ ID No. 1.
5. The nucleotide sequence of embodiment 2, wherein the sequence is at least 91%, at least 92%, at least 93%, at least 94%, at least 95%, at least 96%, at least 97%, at least 98% or at least 99% to the sequence set forth as SEQ ID No. 2.
6. An expression vector comprising the nucleotide sequence described in any one of the embodiments 1 to 5.
7. A host cell comprising the expression vector of embodiment 6.
8. A method of producing recombinant host cell comprising-preparing an expression vector comprising the nucleotide sequence described in any one of the embodiments 1 to 5, operably linked to a protein of interest; and transforming a cell with the said expression vector to obtain recombinant host cell.
9. A method of producing heterologous protein or protein of interest, comprising-growing the host cell described above in a culture medium comprising carbon source selected from a group comprising glucose, glycerol, ethanol, acetate and peptone; and replacing the carbon source with component selected from a group comprising glutamate, monosodium glutamate and combination thereof for producing the heterologous protein.
10. The method of the embodiments 9, provides for improved production of the protein when compared to the method of protein production not under the influence of the nucleotide sequence described in any one of the embodiments 1 to 5.
11. A kit comprising the expression vector described in the embodiment 6, optionally along with restriction endonuclease, ligase, buffer, instruction for use and any other components suitable for use in a kit for making and using the expression vector.
12. Use of the nucleotide sequence described in any one of the embodiments 1 to 5 for producing protein of interest or heterologous protein.

13. Use of the expression vector described in the embodiment 6 for producing the protein of interest or heterologous protein.

14. Use of the expression vector described in the embodiment 6 for producing transforming a host cell for expressing heterologous protein.

15. Use of the host cell described in the embodiment 7 for producing the protein of interest or heterologous protein.

It is to be understood that the foregoing description is illustrative not a limitation. While considerable emphasis has been placed herein on particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure, certain aspects have been employed. The examples used herein for such illustration are intended merely to facilitate an understanding of ways in which the embodiments may be practiced and to further enable those of skill in the art to practice the embodiments. Accordingly, following examples should not be construed as limiting the scope of the embodiments herein.

### EXAMPLES

### Example 1: Generation of Pichia pastoris Strains Harbouring Expression Cassettes Containing SEQ ID No. 1 (GDH2 Promoter) and SEQ ID No. 2 (PEPCK Promoter) Linked to the Gene Encoding GFP (Green Fluorescent Protein)

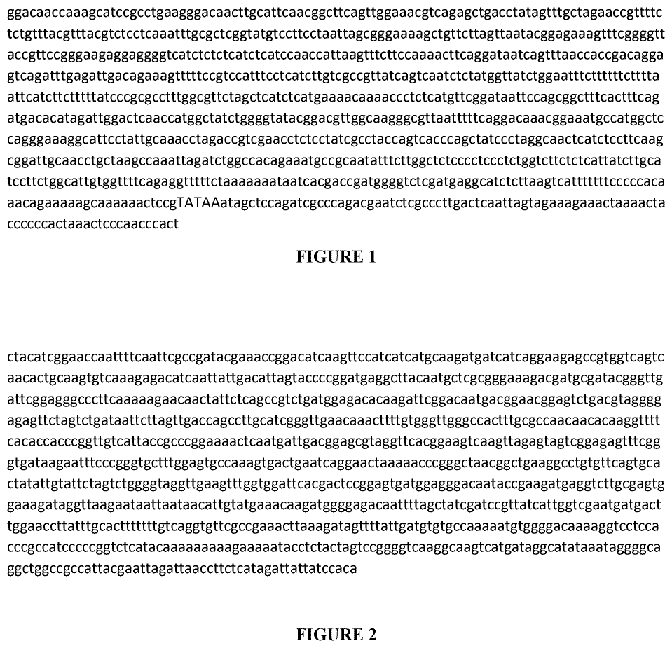

In this example, Pichia pastoris strains harbouring expression cassettes containing Pichia pastoris GDH2 and PEPCK promoters linked to the gene encoding GFP were generated. GFP gene was cloned downstream of 995 bp of GDH2 promoter (FIGS. 1) and 1000 bp of PEPCK promoter (FIG. 2) into pIB3 vector (cat #25452, Addgene, USA) and expressed in Pichia pastoris GS115 strain. For the generation of pGDH2-GFP (pTR1-GFP) construct, 995 bp GDH2 promoter was amplified from GS115 genomic DNA using primer pairs 5′ CCGCTCGAGGGACAACCAAAGCATCC 3′ and 5′ CTCCTTTACTAGTCAGATCTA CCATAGTGGGTTGGGAGTTTAGTGG 3′. GFP gene was amplified using primer pairs 5′ CCACTAAACTCCCAACCCACTATGGTAGATCTGACTAGTAAAGGAG 3′ and 5′ CCCAAG CTTCTAGTGGTGGTGGCTAGCTTTG 3′. The amplified GDH2 and GFP PCR products were then purified and used as templates in the final PCR reaction using primer pairs 5′ CCGCTCGAGGGACAACCAAAGCATCC 3′ and 5′ CCCAAGCTTCTAGTGGTGGTGGC TAGCTTTG 3′. The overlapping product was digested and ligated in pIB3 vector (Addgene plasmid #25452; http://n2t.net/addgene: 25452; RRID: Addgene_25452). The ligation mix was transformed into E. coli DH5a competent cells.

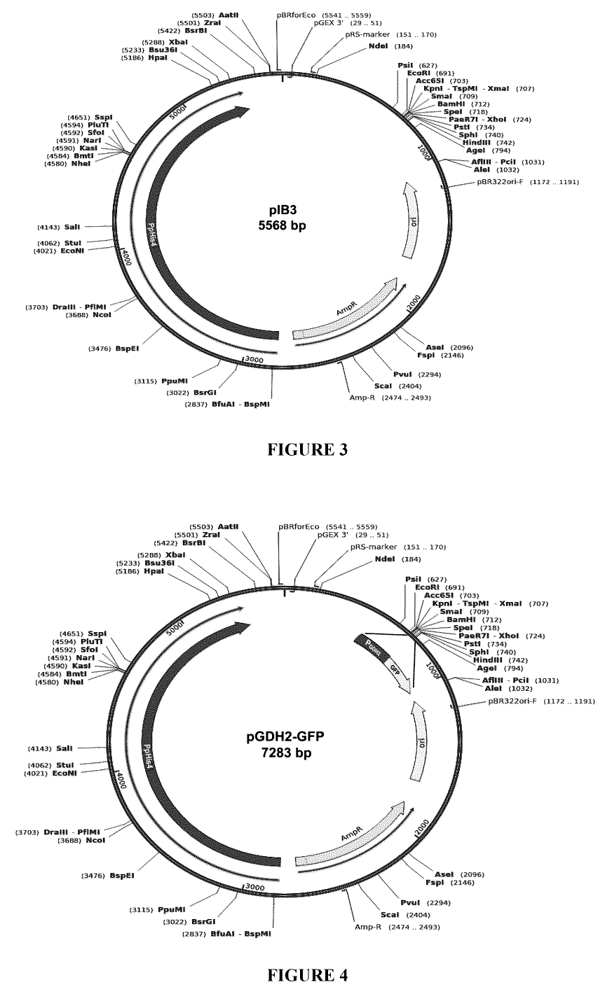

For the generation of PPEPCK-GFP construct, 1 kb PEPCK promoter was amplified from Pichia pastoris GS115 genomic 5′ DNA using primer pairs GGGGTACCCACCCACCCTCAAGTGC 3′ and 5′ CCTTCTCATAGATTATTATCCACAAT GGTAGATCTGACTAGTAAAGGAG 3′. GFP was amplified using primer pairs 5′ CTCCTTTACTAGTCAGATCTACCATTGTGGATAATAATCTATGAGAAGG 3 and 5′ CCCAAGCTTCTAGTGGTGGTGGCTAGCTTTG 3′. The amplified PEPCK and GFP PCR products were then purified and used as templates in the final PCR reaction using primer pairs 5′ GGGGTACCCACCCACCCTCAAGTGC 3′ and 5′ CCCAAGCTTCTAGTGGTGGTGGCT AGCTTTG 3′. The overlapping PCR product was digested and ligated into pIB3 vector (Addgene plasmid #25452; http://n2t.net/addgene: 25452; RRID: Addgene_25452) followed by transformation into E. coli DH5a competent cells. Both the recombinant plasmids were linearized with SalI restriction enzyme and transformed by electroporation into Pichia pastoris GS115 .Recombinant clones were selected by plating the cells on an agar plate containing yeast nitrogen base and dextrose (YNBD) devoid of histidine (YNBD His-). pIB3 vector, and the vector having pGDH2-GFP and pPEPCK-GFP are schematically shown in FIGS. 3,4 and 5, respectively.

### Example 2: Identification of SEQ ID No. 1 (GDH2 Promoter) and SEQ ID No. 2 (PEPCK Promoter) as Glutamate- and Monosodium Glutamate-Inducible Promoters

In this example, GFP expression from GDH2 and PEPCK promoters in cells cultured in media containing different carbon sources we assessed. Cells harbouring P_{GDH2}-GFP and P_{PEPCK}-GFP were cultured in a medium containing yeast nitrogen base (YNB) and glutamate (YNB Glu⁺), YNB containing glucose (YNBD), glycerol (YNBG), ethanol (YNBE) and acetate (YNBA). GFP expression was examined by live cell imaging using confocal microscopy. GFP was expressed at high levels from GDH2 as well as PEPCK promoters in cells cultured in YNB Glu⁺ (FIG. 6), indicating that GDH2 and PEPCK promoters function as glutamate-inducible promoters. In case of P_{PEPCK}-GFP, GFP expression was also detectable in cells cultured in YNBA, YNBE and YNBG (FIG. 6). In this example, GDH2^{His} and PEPCK^{Myc} protein levels from GDH2 and PEPCK promoters in Pichia pastoris GS115 strains cultured in YNB Glu⁺ as well as in YNBD, YNBG, YNBE and YNBA, respectively were examined. GDH2^{His} and PEPCK^{Myc} proteins were detected by western blotting using anti-His and anti-Myc tag antibodies respectively. Expression pattern of GDH2 and PEPCK from GDH2 and PEPCK promoters was similar to GFP expression from GDH2 and PEPCK promoters (FIG. 7B) with highest expression of GDH2^{His} and PEPCK^{Myc} detected in cells cultured in YNB Glu⁺. Thus, glutamate is the best inducer of GDH2 and PEPCK proteins when expressed from GDH2 and PEPCK promoters respectively as well as GFP when expressed from GDH2 and PEPCK promoters.

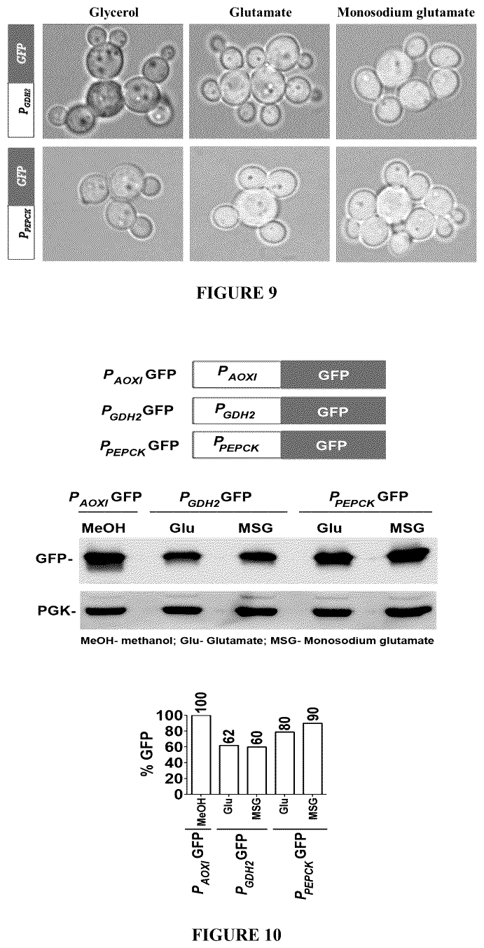

Further, it was observed that there was selective synthesis of GFP under the influence of SEQ ID No. 1 and SEQ ID No. 2. Highest GFP synthesis was observed only under the influence of SEQ ID No. 1 and SEQ ID No.2, respectively when the cells were grown in glutamate containing medium, when compared to other carbon sources such as glucose, glycerol, ethanol and acetate. Monosodium glutamate (MSG) is widely used as a flavour agent in a number of food preparations, inexpensive and is commercially available in large amounts under different trade names such as AJI-NO-MOTO (FIG. 8). In this example, ability of commercially available MSG (AJI-NO-MOTO) to induce GFP expression from Pichia pastoris under the influence of GDH2 promoter (SEQ ID No. 1) and PEPCK promoter (SEQ ID No. 2) were examined. When examined by live cell imaging using confocal microscopy, GFP expression was readily induced by MSG (FIG. 9) indicating that expression of heterologous proteins from GDH2 and PEPCK promoters can be induced by either glutamate or glutamate derivatives such as MSG. Thus, plasmids harbouring SEQ ID No. 1 and SEQ ID No. 2 can be used as Pichia pastoris expression vectors for the expression of heterologous proteins.

### Example 3: Quantification of GFP Expression From Glutamate- and Methanol-Inducible Promoters

In this example, comparison between glutamate-inducible expression of GFP from GDH2 and PEPCK promoters (SEQ ID No. 1 and SEQ ID No. 2) and expression of GFP from methanol-inducible AOXI promoter was carried out by western blotting using anti-GFP antibodies followed by quantitation of the band intensities using ImageJ software and normalized to PGK (phosphoglycerate kinase) control. GFP expression from AOXI promoter was considered as 100%. The results shown in FIG. 10 indicate that GFP expressed from GDH2 and PEPCK promoters is 61±1% and 85±5% of that expressed from AOXI promoter. P_{AOXI}-GFP expression cassette was generated by PCR using routine molecular biology techniques described in the prior art and cloned at the KpnI and XhoI sites of pIB3 vector.

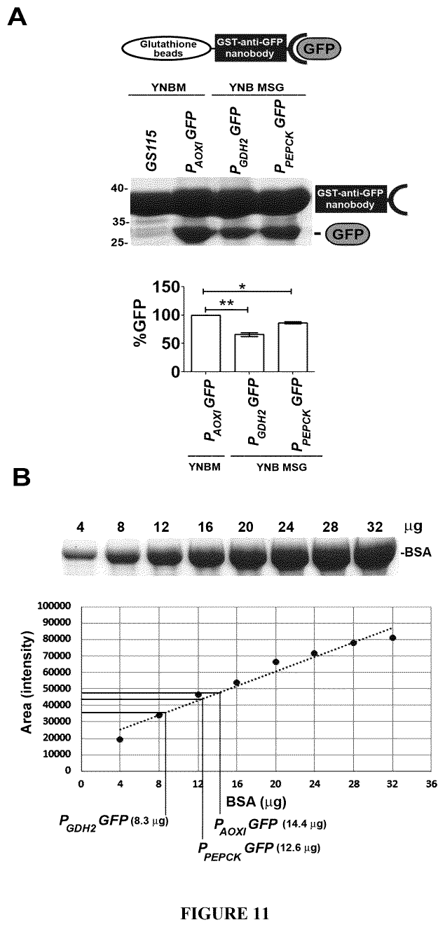

To further quantify GFP expression levels from AOXI promoter (P_{AOXI}), GDH2 promoter (P_{GDH2}) and PEPCK promoter (P_{PEPCK}), GST-tagged anti-GFP nanobodies were used. pGEX6P1-GFP-Nanobody (addgene #61838) was transformed into E. coli cells and expression of the nanobody was induced using IPTG. The nanobody was purified using glutathione agarose beads. Cells harbouring AOXI-GFP expression cassette were cultured in YNB medium containing 1% methanol. Cells harbouring GDH2-GFP and PEPCK-GFP expression cassettes were cultured in YNB medium containing 1% MSG, lysed and incubated with glutathione beads bound to GST-tagged anti-GFP nanobodies. After washing, proteins bound to glutathione beads were analysed by SDS-PAGE and GFP bands were quantified using imageJ software (FIG. 11A). GFP expression from AOXI promoter was considered as 100%. The results shown in FIG. 11A (lower panel) indicate that GFP expressed from GDH2 and PEPCK promoters is about 55% and about 80% of that expressed from AOXI promoter. A BSA standard curve was generated using known amounts of BSA and used for the estimation of amount of GFP expressed from P_{AOXI}, P_{GDH2} and P_{PEPCK}. The results presented in FIG. 11B indicate that 14.4 μg, 12.6 μg and 8.3 μg of GFP is expressed from P_{AOXI}, P_{PEPCK} and P_{GDH2} promoters respectively when lysates were prepared from 20 O.D. cells that were cultured for 24 hours in YNB medium containing MSG (for cell transformed with vector comprising PPEPCK and P_{GDH2} promoters) and methanol (for cell transformed with vector comprising P_{AOXI}).

From the results of this Example, it was noted that, expression of protein under the influence of SEQ ID No. 1 (GDH2 promoter) and SEQ ID No. 2 (PEPCK promoter) in presence of glutamate/MSG provides for following advantages—
Introduction of P_{GDH2}-GFP and P_{PEPCK}-GFP plasmids (expression vector) into the same Pichia pastoris strain can result in GFP expression that is about 1.5 times more than that from methanol inducible, AOXI promoter (60%±90%=150%).
Methanol is toxic and inflammable. MSG is non-toxic and certified as Generally Recognized as Safe (GRAS) by USFDA and is used as common food additive globally. Further, hydrogen peroxide, a by-product of methanol metabolism induces oxidative stress, which can result in the degradation of recombinant proteins. However, Glutamate metabolism does not generate hydrogen peroxide. Thus, using MSG for inducing production of protein of interest by the SEQ ID No. 1 and SEQ ID No. 2 is environmentally friendly and economical and provides for improved protein production without degradants.
Availability of two glutamate-inducible promoters (SEQ ID No. 1 and SEQ ID No. 2) of variable efficacy provides improved opportunity for differential expression of two proteins in the same cell. This provides great utility during metabolic engineering of yeast cells when there is a need to express two different enzymes of metabolic pathway at variable levels.
The described expression system can be used for glutamate-inducible expression of two proteins in the same cell at different molar ratios while assessing protein-protein interactions.

Thus, producing protein of interest under the influence of the said nucleotide sequences (SEQ ID No. 1 and SEQ ID No. 2) is advantageous when compared to producing the protein under the influence of AOXI promoter.

### Example 4: Generation of Glutamate-Inducible Vectors, pTR1-IX, pTR2-IX, pTR1-SX and pTR2-SX for the Intracellular (IX) and Extracellular (SX) Expression of Heterologous Proteins in Pichia pastoris

SEQ ID NO. 1 and SEQ ID NO. 2 described herein are used for the generation of glutamate-inducible vectors containing different selection markers (HIS4, HYG^{R}, ZEO^{R}) as schematically depicted in FIG. 12. pTR1-IX and pTR2-IX are used for expression of protein of interest intracellularly employing SEQ ID No. 1 and SEQ ID NO. 2, respectively. pTRISX and pTR2-SX are used for secretion of the protein of interest into culture medium employing SEQ ID No. 1 and SEQ ID NO. 2 respectively.

Plasmid maps of these vectors are shown in FIG. 13.

### EXAMPLE 5: MSG-Inducible Expression of His-Tagged Human Actin B (HsActinB-His) and His-Tagged SARSCOV-2-Receptor Binding Domain (RBD-His) Using pTR2-IX^{HIS4,HYGR} and pTR2-SX^{HIS4,HYGR} Respectively in Pichia pastoris

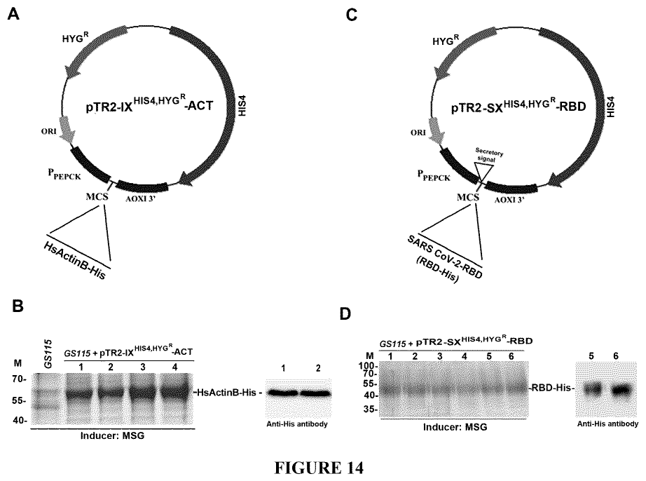

To demonstrate utility of the glutamate-inducible vectors for the production of proteins of biological/commercial importance, genes encoding His-tagged Human actin B (HsActinB-His) and His-tagged SARSCOV-2-receptor binding domain (RBD-His) were cloned into pTR2-IX^{HIS4,HYGR} and pTR2-SX^{HIS4,HYGR} respectively and transformed into Pichia pastoris GS115. Recombinant protein expression was induced by culturing the recombinant yeast cells in YNB medium containing 2% MSG. His-tagged proteins present in cell lysate (HsActinB-His) or in the culture medium (RBD-His) were purified by Ni²⁺ NTA affinity chromatography and visualized in Coomassie Brilliant blue stained SDS polyacrylamide gels (FIG. 14B, D left panels). The identity of the His-tagged proteins was confirmed by western blotting using anti-His tag antibodies (FIG. 14B, D right panels).

### EXAMPLE 6: Enhanced Expression of GFP From Glutamate-/MSG-Inducible Promoters by Co-Transformation of Different Combination of pTR1/pTR2 GFP Expression Vectors

To increase glutamate-/MSG-inducible expression of recombinant proteins, co-transformation strategy wherein two different Glutamate-inducible plasmids expressing GFP carrying different selection markers (a+b, b+c, FIG. 15, A-C) were co-transformed into the same Pichia pastoris GS115 strain and cultured in YNB-1% MSG medium for about 24 hours and GFP expression in the lysates of the recombinant yeast strains was examined by SDS-PAGE followed by western blotting using anti-GFP antibodies. GFP bands were quantified using imageJ software. GFP expression from AOXI promoter was considered as 100%. The results shown in FIG. 15D (upper panel) indicate that GFP expression in cells harbouring a+b and b+c vectors is about 80% and about 120% relative to that in cells expressing GFP from AOXI promoter.

In another experiment, 5.0 O.D. of the cells corresponding to about 1.0 ml of the culture were lysed, lysate was incubated with glutathione beads bound to GST-tagged anti-GFP nanobodies. After washing, proteins bound to glutathione beads were analysed by SDS-PAGE and GFP bands were quantified using imageJ software. A BSA standard curve was generated using known amounts of BSA and used for the estimation of amount of GFP expressed from P_{AOXI} and b+c vectors. Cell lysate of GS115 cultured in YNB-MSG served as control (FIG. 15E). The results indicate that 5.9 μg and 6.29 μg of GFP is expressed from P_{AOXI} and P_{PEPCK} promoters (b+c vectors) respectively. Thus, GFP expression in cells harbouring b+c vectors surpassed that of cells expressing GFP from P_{AOXI} vectors.

### EXAMPLE 7: Comparison of His-Tagged SARSCOV-2RBD (RBD-His) Levels in Pichia pastoris Expressing RBD-His From pTR2-SX^{HIS4,HYGR} Alone or pTR2-SX^{HIS4,HYGR} and pTR2-SX^{ZEOR}

The procedure followed in EXAMPLE 6, was adopted to increase RBD-His levels by co-transformation of Pichia pastoris GS115 with pTR2-SX^{HIS4,HYGR} and pTR2-SX^{ZEOR} expressing GFP (FIG. 16A,B). Pichia pastoris strain in which RBD-His was expressed from both pTR2-SX^{HIS4,HYGR} and pTR2-SX^{ZEOR} which was designated as a+b. Recombinant strains were cultured in YNB-2% MSG medium for about 24 hours and RBD-His in the culture medium was bound to Ni²⁺NTA agarose beads and subjected to SDS-PAGE followed by western blotting using anti-His antibodies. RBD-His bands were quantified using imageJ software. RBD-His expression from cells transformed with pTR2-SX^{HIS4,HYGR} alone (a) was considered as 100%. The results obtained from four different recombinant a+b clones are presented in FIG. 16C. RBD-His expression in a+b clones varied between 200% and 124% relative to that in a (FIG. 16C right panel).

The results under Examples 6 and 7 indicate that intracellular as well as extracellular expression of heterologous proteins is enhanced by co-transformation of two different glutamate-inducible plasmids into the same Pichia pastoris strain.

### EXAMPLE 8: Assembling the Kit

The expression vectors described in the present disclosure were provided in suitable vials independently, capable of maintaining activity of the vectors until use. The vials containing the expression vectors were assembled with additional vials comprising restriction endonuclease, ligase, buffer and selection markers, respectively and an instruction manual providing detailed information about cloning of gene of interest to the expression vector provided in the vials employing the restriction endonuclease, the ligase and buffer provided in the kit.

Optionally, the kit was assembled to contain Pichia pastoris strains for both intracellular and secreted expression.
The kit forms one complete package enabling improved production of the protein of interest or heterologous protein employing the expression vectors provided therein.

We claim:

1. A Nucleotide Sequence selected from
a sequence having at least 90% identity to sequence set forth as SEQ ID No. 1; or
a sequence having at least 90% identity to sequence set forth as SEQ ID No. 2.

2. (canceled)

3. (canceled)

4. The nucleotide sequence as claimed in claim 1, wherein the sequence is the sequence set forth as SEQ ID No. 1.

5. The nucleotide sequence as claimed in claim 1, wherein the sequence is the sequence set forth as SEQ ID No. 2.

6. An expression vector comprising a nucleotide sequence as claimed in claim 1.

7. The expression vector as claimed in claim 6, comprising the nucleotide sequence set for SEQ ID No. 1 or SEQ ID No. 2.

8. The expression vector as claimed in claim 6, comprising a selection marker sequence, a cloning site, an enhancer sequence, a termination sequence, a signal peptide sequence or any combination thereof.

9. The expression vector as claimed in claim 6, wherein the vector is cloned with a gene encoding protein downstream of the SEQ ID No. 1 or the SEQ ID No. 2.

10. The expression vector as claimed in claim 9, wherein the gene encodes a protein selected from the group consisting of a toxin, an antibody, a hormone, an enzyme, a growth factor, cytokine, a structural protein, an immunogenic protein, a vaccine protein, a fusion protein, a chimeric protein, a membrane protein, a non-membrane protein and a cell signalling protein.

11. A host cell comprising the expression vector as claimed in claim 6.

12. The host cell as claimed in claim 11, wherein the host cell comprises a combination of the expression vectors comprising SEQ ID No. 1 and SEQ ID No. 2 with the same or distinct marker.

13. The host cell as claimed in claim 11, wherein the host cell is eukaryotic.

14. The host cell as claimed in claim 11, wherein the host cell is selected from the group consisting of Pichia pastoris, Scheffersomyces stipitis, Candida albicans, Candida maltosa, Candida shehatae, Candida glabrata, Candida reukaufii, Candida utilis, Debaryomyces hansenii, Kluyveromyces lactis, Kluyveromyces marxianus, Lodderomyces elongisporus, Meyerozyma guilliermondii, Pichia capsulata, Yarrowia lipolytica, Rhodotorula rubra, and Trichosporon beigel.

15. A method of producing the a host cell, said method comprising transforming the host cell with the expression vector as claimed in claim 6.

16. A method of producing heterologous protein, said method comprising
a) growing the host cell as claimed in claim 11 in a culture medium comprising a carbon source selected from the group consisting of glucose, glycerol, ethanol, acetate and peptone; and
b) replacing the carbon source with a component selected from the group consisting of glutamate, monosodium glutamate and combinations thereof for producing the heterologous protein.

17. The method as claimed in claim 16, wherein during the growth of the host cell in step a) there is no expression of the heterologous protein.

18. The method as claimed in claim 16, wherein during the step b), the heterologous protein is expressed at an increased level under the influence of a component selected from the group consisting of glutamate, monosodium glutamate and combinations thereof.

19. The method as clamed in claim 16, wherein the method further comprises recovering and purifying the heterologous protein.

20. A kit comprising the expression vector as claimed in claim 6, optionally along with a restriction endonuclease, ligase, or a buffer.

21. (canceled)

22. (canceled)
